(19) United States
(12) Patent Application Publication
(10) Pub. No.: US 2009/0105612 A1
(43) Pub. Date: Apr. 23, 2009

(54) Lancing device end cap with pressure-actuated surface features

(75) Inventors: Christopher J. Ruf, Marietta, GA (US); Stephen J. Flynn, Peachtree City, GA (US); Richard W. LeVaughn, Newnan, GA (US); Gwenn E. Kennedy, Ellenwood, GA (US); Michael V. Lipoma, Villa Rica, GA (US); Juan D. Salleras, Alpharetta, GA (US); Jac Holzaepfel, Newnan, GA (US); Sylvia Ramos, Smyrna, GA (US)

Correspondence Address: GARDNER GROFF GREENWALD & VILLANUEVA. PC, 2018 POWERS FERRY ROAD, SUITE 800, ATLANTA, GA, 30339, US

(73) Assignee: FACET TECHNOLOGIES, LLC, Marietta, GA (US)

(21) Appl. No.: 10/572,036

(22) PCT Filed: Jul. 28, 2004
(86) PCT No.: PCT/US04/24279

§ 371 Date: Mar. 17, 2008

Related U.S. Application Data
(60) Provisional application No. 60/503,862, filed on Sep. 18, 2003.

(51) Int. Cl. A61B 5/151

(52) U.S. Cl. 600/583

(57) ABSTRACT

An endcap for a lancing device, the endcap including a contact face defining an opening for passage of a sharp lancet tip, wherein the contact face includes one or more surface features for compression and/or twisting against the skin around a sampling site to enhance sample collection and/or prevent premature wound closure.

### CROSS-REFERENCE TO RELATED APPLICATION

This application claims the benefit of U.S. Provisional Patent Application Ser. No. 60/503,862, filed Sep. 18, 2003, the entirety of which is hereby incorporated herein by reference for all purposes.

### TECHNICAL FIELD

The present invention relates generally to medical devices and procedures, and more particularly to lancing devices for sampling blood or other bodily fluids. The invention more particularly relates to an end cap for use in conjunction with a lancing device that allows for the collection of an increased volume of blood or bodily fluid to be sampled.

### BACKGROUND OF THE INVENTION

Many medical procedures require puncturing of the skin, and sometimes underlying tissues, of a human or animal subject. For example, a sharp lancet tip is commonly used to puncture the subject's skin for sampling of blood or other body fluid, as for example in blood glucose monitoring by diabetics. Generally, lancet blades or needles are much thinner than traditional hypodermic syringe needles, and therefore result in less pain to the patient. A lancet having a sharp lancing tip typically is mounted, permanently or releasably, to the drive mechanism of a lancing device. The drive mechanism includes one or more springs, cams, motors, or other mechanism(s) for moving the lancing tip from a retracted position shielded within a housing of the lancing device to an extended position outside of the housing for lancing the subject's skin. The lancing device may also include a cocking mechanism for arming the drive mechanism, and/or a triggering mechanism for firing the device. The housing of the lancing device typically includes an endcap with an opening through which the lancing tip extends in its extended position. The endcap may be releasably or permanently attached to the remainder of the housing, or can comprise an integral part of the housing.

One common problem that has been found to exist with many known lancets and lancing devices is the closure of the puncture site before an adequate volume of blood has been collected. Premature closure of the puncture requires additional blood to be drawn from the patient, often in a separate and sometimes less desirable location, resulting in an increase in pain and stress to the subject. Costs also increase, as multiple lancets are required to acquire an adequate sample. This is particularly the case when alternate lancing sites other than the fingertips, such as for example the forearm or earlobe, are lanced. Lancing at such alternate lancing sites may be desirable because repeated lancing of the fingertips can result in callous formation, leading to less consistent sampling and decreased sensitivity of the fingertips. In addition, the fingertips have a greater density of nerve endings than alternate lancing sites, resulting in greater pain sensation from fingertip lancing than lancing at alternate sites. To increase patient compliance with a prescribed sampling regimen, it is desirable to minimize pain resulting from the lancing operation. However, because alternate sites typically contain a lower density of capillaries or a deeper capillary bed than the fingertips, alternate lancing sites often yield insufficient sample sizes or are prone to premature closure.

Attempts have been made to provide lancing devices that inhibit premature closure of the puncture site and yield increased sample size. Such attempts have included the use of a stimulator member slideably mounted to the housing for cyclically pumping around the puncture site to “milk” the wound. Although utilizing the aforementioned pumping sequence may allow a larger sample to be collected before closure of the puncture site, occasionally a sample is still insufficient, and another lancing operation to collect blood or analytic fluid is required. Additionally, the repeated application and release of pressure from such pumping can result in contact between the sample and the lancing device, smearing and/or contaminating the sample and necessitating another lancing operation, and/or can lead to bruising of the tissue surrounding the lancing site. Also, the provision of a translationally mounted stimulator member results in increased complexity and cost of the lancing device.

Thus it can be seen that needs exist for improved lancing devices and methods to facilitate increased sample volume of blood or other analytic fluid, and to prevent premature closure of the sampling site. Needs further exist for such mechanisms and methods that are readily adaptable to current lancing devices and procedures, and which can be utilized for lancing at fingertip as well as alternate sample collection sites.

### SUMMARY OF THE INVENTION

In example forms, the present invention is an endcap for a lancing device that enables collection of an increased volume of blood or other body fluid to be sampled from a lancing site of a human or animal subject. The device of the present invention is suitable for use at fingertip lancing sites, but is also well suited for use at alternate lancing sites such as the forearm and/or earlobe. In further embodiments, the invention is a sampling method for increasing collected sample size of blood or other body fluid from a lancing site.

In one aspect, the present invention is an endcap for a lancing device, the endcap preferably having a contact face defining an opening for passage of a sharp lancet tip, and one or more surface features projecting from the contact face for contact against the skin around a sampling site to enhance sample collection.

In another aspect, the invention is a lancing device for collecting a sample of body fluid from a sampling site on the skin of a subject. The lancing device preferably includes a lancet and a housing comprising an endcap, the lancet being movable between a first position within the housing and a second position wherein at least a sharp tip portion of the lancet extends through an opening in the endcap. The endcap preferably has a contact face surrounding the opening and includes at least one surface feature projecting outwardly from the contact face.

In still another aspect, the invention is a method of collecting a sample of blood or other body fluid, the method including the steps of lancing the skin of a subject at a lancing site with a lancing device having a contact face including one or more surface features projecting therefrom; and applying pressure between the contact face and the skin surrounding the lancing site to enhance sample collection.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a perspective view of an endcap for a lancing device according to one example embodiment of the present invention.

FIG. 2 shows a perspective view of an endcap for a lancing device according to another example embodiment of the present invention.

FIGS. 3a and 3b show perspective views of endcaps for a lancing device according to still further example embodiments of the present invention.

FIG. 4 shows a perspective view of an endcap for a lancing device according to another example embodiment of the present invention.

FIG. 5 shows a perspective view of an endcap for a lancing device according to still another example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

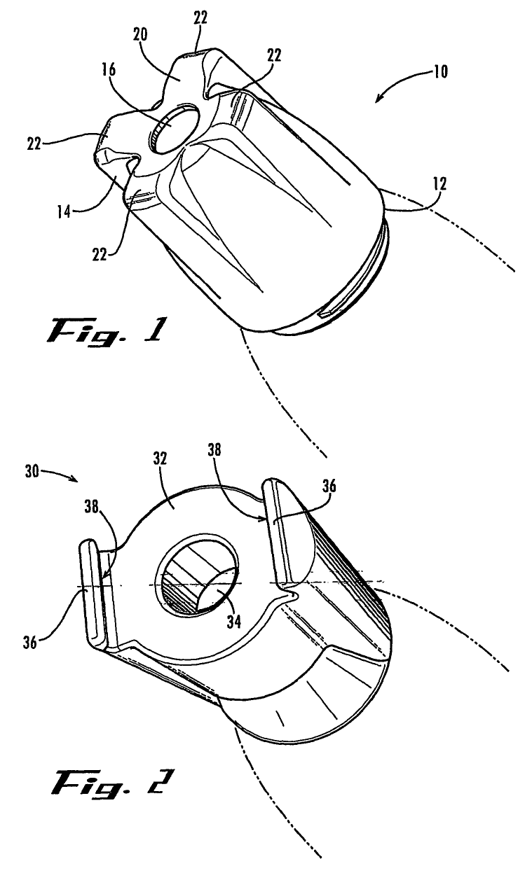

With reference now to the drawing figures, FIG. 1 shows one example embodiment of an endcap 10 for a lancing device according to the present invention. The endcap 10 preferably comprises a first end 12 for releasable or permanent attachment to the remainder of a housing of a lancing device. Alternatively, the endcap forms an integral part of the housing of the lancing device. The endcap 10 can be adapted for use with any of a variety of standard, commercially-available or later-developed lancing devices, by appropriate configuration of coupling or attachment features of the first end 12. The second end 14 of the endcap 10 preferably defines the opening 16 through which the sharp lancet tip projects in its extended position to penetrate the skin at the intended lancing site.

In the depicted embodiment, the opening 16 is a generally circular opening formed at or near the center of the second end 14 of the endcap 10. In alternate embodiments, the opening is a slot or an opening of triangular, rectangular, polygonal or other shape. The second end 14 of the endcap 10 preferably comprises a concave contact face 20, with one or more surface features formed thereon. For example, in the depicted embodiment, the surface features comprise one or more raised ribs or projections 22 (four projections arranged in a cross-shaped configuration are shown) extending outwardly from the contact face 20.

In a preferred method of operation according to the present invention, after firing the lancing device and piercing the skin at the sampling site, the user leaves the contact face of the endcap in contact with the skin around the lancing site, and applies a steady, continuous pressure against the skin with the contact face. Optionally, the lancing device may be twisted and/or rocked with the contact face of the endcap pressed against the skin. In an alternate method, discontinuous pressure is applied to the skin around the lancing site by cyclically pumping the contact face against the skin. With the endcap embodiment of FIG. 1, the raised ribs 22 apply pressure around the puncture site while the spaces between the raised ribs allow subcutaneous blood or other body fluid to flow toward the puncture site, thereby enhancing collection of increased volume of blood or other body fluid to be sampled and helping to prevent premature closure of the puncture site.

FIG. 2 shows an endcap 30 for a lancing device according to another embodiment of the present invention. The contact face 32 of this endcap defines a generally circular central opening 34 for allowing passage of the sharp lancet tip. One or more compression ribs 36 preferably project outwardly from the contact face 32 to engage and tension the skin as pressure is applied, opening the wound to enhance bloodflow and prevent premature closure, thereby facilitating collection of an increased volume of blood or other body fluid to be sampled. In the depicted embodiment, first and second compression ribs 36 are positioned on opposite sides of the opening 34. A curved or angled inwardly-recessed channel 38 is preferably formed at the intersection of each rib 36 with the contact face, into which the skin may bulge upon tensioning of the skin by application of pressure between the contact face against the tissue around the lancing site.

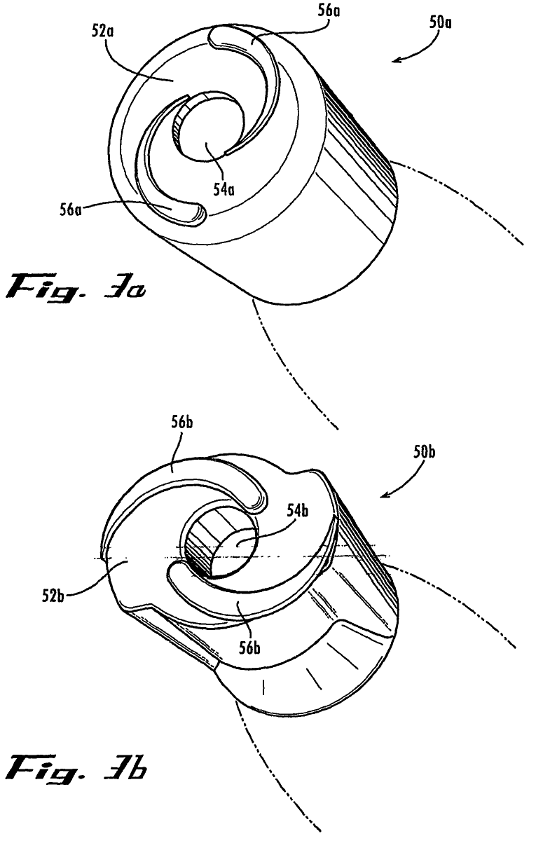

FIGS. 3a and 3b show two related embodiments of endcaps 50 (corresponding elements are indicated with reference numbers followed by the letter “a” for the embodiment of FIG. 3a, and by the letter “b” for the embodiment of FIG. 3b) for a lancing device according to still further embodiments of the present invention. The contact face 52 of these endcaps defines a generally circular central opening 54 for passing the sharp lancet tip. The contact face 52 preferably comprises one or more helical vanes 56 spiraling inwardly from the periphery of the contact face 52 toward the opening 54. Optionally, the vanes 56 are wider and/or deeper at their inner terminus, and taper down along their length to a narrower and/or shallower dimension at their outer terminus, as shown in the embodiment of FIG. 3b; or are wider and/or deeper at their outer terminus, and taper down along their length to a narrower and/or shallower dimension at their inner terminus, as shown in the embodiment of FIG. 3a. By applying pressure and twisting the contact face 52 against the skin, the vanes 56 stimulate blood-flow in surrounding tissue and direct subcutaneous blood toward the wound at the lancing site, thereby enhancing sample collection.

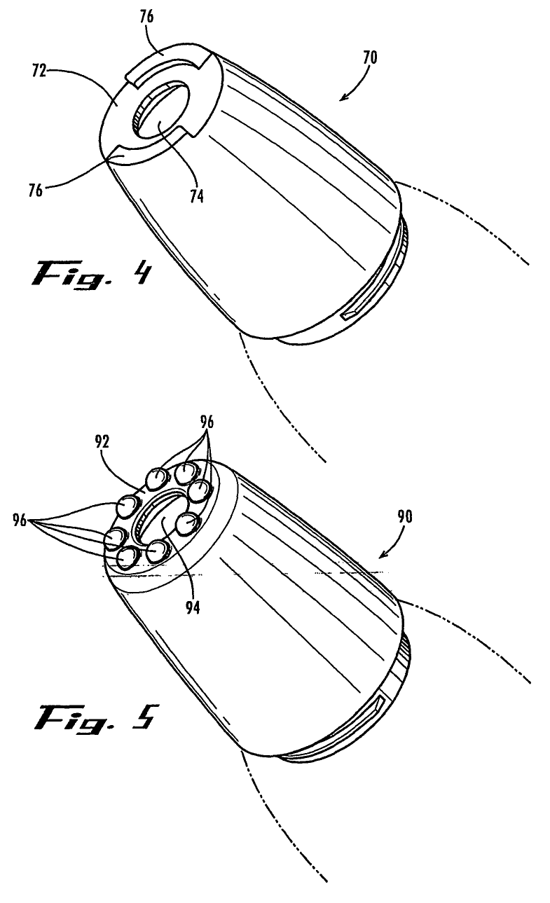

FIG. 4 shows an endcap 70 for a lancing device according to another embodiment of the present invention. The contact face 72 of this endcap defines a generally circular central opening 74 for passing the sharp lancet tip. The contact face 72 preferably comprises one or more raised fins 76 spaced a distance away from the opening 74. For example, in the depicted embodiment, first and second raised fins 76 project forward from the outer circumference of the contact face 72 on opposite sides of the opening 74. Optionally, the raised fins 72 are inclined relative to the contact face 72, their distal faces forming a ramp that is pressed against the skin around the lancing site. After lancing the skin, the lancing device is twisted in a clockwise and/or counterclockwise manner, with the contact face 72 pressed against the skin to prevent premature closure of the wound and to enhance collection of an increased volume of blood or other body fluid to be sampled.

FIG. 5 shows an endcap 90 for a lancing device according to still another embodiment of the present invention. This endcap includes a contact face 92 with a generally circular central opening 94 formed therein. One or more raised projections 96 are preferably provided, projecting outwardly from the contact face 92 around at least a portion of the circumference of the opening 94. In the depicted embodiment, each projection 96 is a generally hemispherical dome-shaped projection. After lancing, the lancing device is pressed against the skin around the lancing site and twisted with the projections 96 engaging the skin to help prevent premature closure of the puncture site and enhance sample collection.

While the invention has been described with reference to preferred and example embodiments, it will be understood by those skilled in the art that a variety of modifications, additions and deletions are within the scope of the invention, as defined by the following claims.

What is claimed is:

1. An endcap for a lancing device, said endcap comprising a contact face defining an opening for passage of a sharp lancet tip, said endcap further comprising one or more surface features projecting from the contact face for contact against the skin around a sampling site to enhance sample collection.

2. The endcap of claim 1, wherein said one or more surface features comprise one or more raised ribs.

3. The endcap of claim 2, comprising four raised ribs in a cross-shaped array.

4. The endcap of claim 1, wherein said one or more surface features comprise first and second compression ribs positioned on opposite sides of the opening.

5. The endcap of claim 4, wherein a curved or angled channel is formed at the intersection of the compression ribs with the contact face.

6. The endcap of claim 1, wherein said one or more surface features comprise one or more helical vanes spiraling inwardly from the periphery of the contact face toward the opening.

7. The endcap of claim 6, wherein each helical vane has a larger dimension at its inner terminus, and tapers down along its length to a smaller dimension at its outer terminus.

8. The endcap of claim 6, wherein each helical vane has a larger dimension at its outer terminus, and tapers down along its length to a smaller dimension at its inner terminus.

9. The endcap of claim 1, wherein said one or more surface features comprise one or more raised fins spaced a distance away from the opening.

10. The endcap of claim 1, wherein said one or more surface features comprise one or more raised projections, projecting distally from the contact face around at least a portion of the circumference of the opening.

11. The endcap of claim 10, wherein each projection is a generally hemispherical dome-shaped projection.

12. The endcap of claim 1, wherein said contact face is concave.

13. A lancing device for collecting a sample of body fluid from a sampling site on the skin of a subject, said lancing device comprising a lancet and a housing comprising an endcap, the lancet being movable between a first position within the housing and a second position wherein at least a sharp tip portion of the lancet extends through an opening in the endcap, wherein the endcap comprises a contact face surrounding the opening and at least one surface feature projecting outwardly from the contact face.

14. The lancing device of claim 13, wherein the at least one surface feature comprises at least one raised rib extending at least partially across said contact face.

15. The lancing device of claim 14, wherein the at least one raised rib comprises four raised ribs in a cross-shaped array.

16. The lancing device of claim 14, wherein a recessed channel is formed at the intersection of the at least one raised rib and the contact face.

17. The lancing device of claim 13, wherein the at least one surface feature comprises at least one helical vane spiraling inwardly from the periphery of the contact face toward the opening.

18. The lancing device of claim 13, wherein the at least one surface feature comprises at least one inclined ramp projecting outwardly from the contact face.

19. The lancing device of claim 13, wherein the at least one surface feature comprises a plurality of generally hemispherical dome-shaped projections.

20. The lancing device of claim 13, wherein the contact face is concave.

21. A method of collecting a sample of blood or other body fluid, comprising:
lancing the skin of a subject at a lancing site with a lancing device having a contact face including one or more surface features projecting therefrom; and
applying pressure between the one or more surface features on the contact face and the skin surrounding the lancing site to enhance sample collection.

22. The method of claim 21, further comprising twisting the contact face against the skin.
